(19) United States
(12) Patent Application Publication
(10) Pub. No.: US 2010/0150973 A1
(43) Pub. Date: Jun. 17, 2010

(54) Granules for use in Formulations of Personal Care and/or Home Care Products and Products Containing Said Granules

(76) Inventor: Patricia Moura Dias, Santos (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/444,925

(22) PCT Filed: Mar. 20, 2007
(86) PCT No.: PCT/BR07/00069

§ 371 Date: Feb. 1, 2010

(30) Foreign Application Priority Data
Oct. 11, 2006 (BR) PI0604286-4

(51) Int. Cl. A61K 8/02; A61K 8/73; B32B 5/00; A61Q 11/00; A61Q 5/12; C11D 3/22; C11D 3/02

(52) U.S. Cl. 424/401; 514/770; 428/402; 510/474; 510/507

(57) ABSTRACT

The present invention relates to a granule for use in formulations of personal care and/or home care products having liquid to creamy (or pasty) consistency, the granule or granules being specially suitable for use as visual agents, delivery systems, exfoliating and/or abrasive agents in these products. The object of the present invention is to provide granules consisting of clay or clay and starch, which maintain their physical integrity in formulations of personal care and/or home care products with liquid to creamy (or pasty) consistency. This object is achieved by a granule consisting of the combination of clay and starch, wherein at least one portion of clay is thermally treated, with modification of the crystalline structure of at least one fraction of the treated clay. The object is also achieved by a granule consisting of clay, wherein at least one portion of the clay is thermally treated, with alteration of the crystalline structure of at least one fraction of the clay.

### FIELD OF THE INVENTION

The present invention relates to granules for use in formulations of personal care and/or home care products having liquid to creamy (or pasty) consistency. The granules according to the present invention are specially suitable for use as visual agents, delivery systems, exfoliating and/or abrasive agents.

### DESCRIPTION OF THE PRIOR-ART

Clay is a natural material, product from the alteration of volcanic rocks and/or ashes in the presence of water. These alterations occur close to the earth's surface and, for this reason, heat and pressure above the ambient may be involved. Synthetic clays also exist and their general properties and behavior are the same as in the natural clays.

Clay and clay mineral are terms that designate different materials. Clay is a mixture of components, the relative proportion of which determine its properties, being mainly formed by clay minerals, but it may also contain other materials that are not clay minerals (for example, quartz, metallic oxides, carbonates and sulfates), organic matter (humus) and soluble salts. It is the clay mineral predominating in the clay that confers to it its major properties, such as plasticity, hardness when burned, cation exchange capacity, molecular sieve, thixotropy, among others. The terms “clay” and “clay mineral” tend to be used as synonyms, although they designate different materials.

Clay minerals are composed by tetrahedral and octahedral structural units. The SiO₄ tetrahedra share vertices with each other and are arranged in tetrahedral sheets. The octahedral units are ideally composed by Mg(OH)₂ and/or Al(OH)₃, sharing edges with each other, generating octahedral sheets. The dimensions of the tetrahedral and octahedral sheets are similar, allowing them to share oxygen atoms with each other. Oxygen sharing is observed between a tetrahedral and a octahedral sheet, a structure called T:O (tetrahedra:octahedra) characteristic of the kaolinite group. Structures in which an octahedral sheet shares oxygens with two tetrahedra sheets, one above and another below, like in a sandwich, are called T:O:T structures (tetrahedra:octahedra:tetrahedra). T:O:T structures are the most abundant type in nature and in synthetic clays.

Clays are called phyllosilicates (“sheet silicates”) because of the dimensions of their layers, with the width and length much larger than the height. The dimensions of the layers depend on a series of factors involved in their synthesis, but an aspect is repeated: the stacking of consecutive layers, one over the other, separated by hydrated cations and/or water molecules. The number of stacked layers may vary from very few to dozens, and the energy involved in maintaining this structure may vary from weak to very strong.

During the clays synthesis, Mg²⁺, Al³⁺ and Si⁴⁺ cations may be replaced with others of similar size, but of lower valence. This replacement depends on the environment conditions and the availability of cations present at the moment of the clay synthesis. These replacements may occur only in the tetrahedral sheet, only in the octahedral sheet or in both, they may be extensive or restricted to a few cations. When present, this isomorphic substitution generates in the lamellar structure a charge deficiency, which must be neutralized by cations. The neutralizing cations are associated with the clay structure and are found in their hydrated form between the layers and outside them. The most commonly found cations neutralizing the clay layers are the alkaline cations and the alkaline earth cations, particularly, Na⁺, K⁺, Mg²⁺ and Ca²⁺.

In clays with an intermediate degree of isomorphic substitution, as in the group of smectites and in the group of vermiculites, the interlamellar neutralizing cations may be replaced with others, either inorganic and/or organic. Another important property of clays is its ability to increase the space between adjacent layers by the hydration of interlamellar cations or the intercalation of voluminous species. The inorganic interlamellar cations and the clay structure itself have large affinity for water, and as a function of their hydration, the disaggregation of clay particles in water is observed.

In some cases, it is possible to turn the phyllosilicate resistant to the hydration and disaggregation processes. Even when it has a dry aspect, clay has water molecules associated with its structure, particularly interlamellar water, hydrating the neutralizing cations. When heated up to about 300° C., the gradual loss of water molecules associated with clay occurs. At first, the molecules externally associated with the lamellar structure are lost, and later, as the temperature increases, the water molecules in the interlamellar spaces are lost as well as those hydrating the cations present therein.

If the temperature rises above 300° C., other thermodynamic processes may occur, changing the physico-chemical characteristics of the clay. At higher temperatures, the structural hydroxyls, located in the octahedral sheets start to react with each other. Every two hydroxyls generate a water molecule, which is lost, and an oxygen bridge. The temperature at which this process begins is usually around 600° C.

As the temperature gradually rises, more structural water molecules are lost and oxygen bridges are formed. This process is called dehydroxilation, the loss of hydroxyls by heating. Dehydroxilation changes the crystalline structure of clay; it may be a complete or partial process only depending on the heating temperature and time: high temperatures for long periods of time tend to generate completely dehydroxilated materials. However, the temperature cannot be indefinitely raised, because after the complete dehydroxilation of clay, a vitrification process may begin. In general, up to 1,000° C., there is no risk of vitrification of the material. Above this temperature, the clay mineral behavior becomes a function of its structural composition and characteristics.

As stated above, dehydroxilation changes the physico-chemical characteristics of the clay, particularly with regard to its mechanical and chemical resistance. The change in the properties of thermally treated clay confers lower hydrophilicity to the aluminosilicate and, thus, greater resistance to the hydration and disaggregation processes, when in water or media that is rich in this solvent.

Clays are commonly used as raw materials in personal care and home care products with different functions, such as: rheology modifiers, fillers, detergent softener agents, active substances and/or fragrance encapsulators or as visual elements in its granulate form, among several others.

EP 816.484 discloses solid detergent compositions, such as bars, powders and granules. The object of this composition is to provide low loss of perfume by evaporation during its production at high temperatures, which is achieved due to adsorption of the perfume and the viscosity agent in the mineral carrier.

US 2004/0002438 discloses liquid compositions containing surfactants and clay particles, among them bentonite, which remain in suspension acting, for instance, as exfoliating and sequestrating agents. Document US 2003/0134771 describes liquid aqueous compositions for fabric conditioning containing surfactants and clay particles such as suspended bentonite. According to said document, clays act as softeners, delivery systems for perfumes and stabilization agents. The liquid formulation is preferably an emulsion. However, neither of the US documents cited above discloses or suggest clay granules with the features specifically defined in the present application.

The prior art does not deal with granules having high integrity in fluid media and which, in case of disintegration, do so under the action of friction at the moment the product that contains them is used. Granules which exhibit high integrity may act as visual agents, delivery systems, exfoliating and/or abrasive agents. High integrity means maintaining the physical aspect of the granule, without its complete or partial disaggregation when it is placed in fluid media, containing or not containing water. As stated, if happens, grain disaggregation only occurs when friction is employed.

### OBJECTS OF THE INVENTION

The object of the present invention is to provide granules consisting of clay or clay and starch, which maintain their integrity in formulations of personal care and/or home care products with fluid, liquid to creamy (or pasty) consistency, to act as visual agents, delivery systems, exfoliating and/or abrasive agents. The disintegration of said granules, if so, occurs at the moment the end product is used by employing friction. Granule integrity means the maintenance of its physical aspect, without complete or partial disaggregation thereof, during the entire preparation process of formulations in which the granules will be used, and these formulations may or may not contain water at variable concentrations.

### SUMMARY OF THE INVENTION

The object of the present invention is achieved by a granule consisting of clay or clay and starch for use in formulations of personal care and/or home care products with liquid to creamy or pasty consistency. At least one portion of the clay must be thermally treated, with alteration of at least one fraction of the crystalline structure of the treated material. Thermal treatment means submitting the clay to heating during such period of time and temperature so as to enable that at least one fraction of the clay undergoes dehydroxilation and, therefore, changes its crystalline structure. Dehydroxilation alters the physico-chemical properties of the material, imparting greater resistance to clay against disaggregation processes commonly undergone by them in environments containing water.

The present invention also encompasses products for personal care and/or home care containing said granules.

### DETAILED DESCRIPTION OF THE INVENTION

In a first embodiment of the invention, a granule is provided for use in formulations of personal care and/or home care products with liquid to creamy (or pasty) consistency, wherein the granule consisting of a combination of clay and starch. In this embodiment, at least one portion of the clay composing the granule is thermally treated so that at least part of the treated clay has its crystalline structure altered by the thermal treatment. Thermal treatment means submitting clay to heating during such period of time and temperature so as to enable that at least one fraction of the clay undergoes dehydroxilation and, therefore, changes its crystalline structure. The proportion of thermally-treated clay in the granules of the present invention varies from 0.5% to 100% by weight based on the total weight of the granule composition.

The thermal treatment may be carried out during periods of time that vary from 30 minutes to 12 hours at temperatures varying between 600° C. and 1,200° C.; preferably temperatures between 700° C. and 900° C., and periods of time between 1 hour and 8 hours, the most suitable periods of time being between 1 hour and 5 hours.

According to this embodiment of the invention, the ratio by weight between clay and starch may be quite variable, and it may be from about 1% to about 99% by weight of clay based on the total weight of the composition or, correspondingly, from about 1% to about 99% by weight of starch based on the total weight of the composition. The clay used may be of any type, such as bentonite, vermiculite, kaolin, talc or mica. Preferably, bentonite is used, may it be natural sodium bentonite, semi-sodium bentonite, calcium bentonite, activated sodium bentonite or polycationic bentonite. More particularly, as an example, the following commercially available bentonites are used: activated sodium bentonite (Brasgel Aço) from Bentonit União Nordeste Ltda. (BR), natural sodium bentonite (Volclay) from American Colloid (EUA), natural sodium bentonite (Argel CN 40) from Buntech (BR), natural semi-sodium bentonite (Argel 10) from Buntech (BR).

Starch may be selected from corn (maize) starch, cassava (manioc) starch or mixtures thereof, and it may be natural starch, modified starch or mixtures thereof. Preferably, pre-gelatinized corn starch is used. Pre-gelatinized corn starch, in contact with the water used in the granulation process, forms a material with an aspect similar to that of gelatin, which acts as aggregating agent of the powdered raw material and helps in its conformation in the form of granule. More specifically, for instance, starches that may be used are the following commercially available products: anionic corn starch, cross-linked Snow Flake 6800 starch, amphoteric corn starch, and pregelatinized Amidex G 2100 starch, all from Corn Products Brasil.

In a second embodiment of the invention, a granule is provided for use in formulations of personal care and/or home care products of liquid to creamy (or pasty) consistency, wherein the granule is composed of clay. In this embodiment, at least one portion of the clay that composes the granule is thermally treated, wherein said treatment changes the crystalline structure of at least part of the treated clay. As already mentioned above, thermal treatment means submitting the clay to heating during such period of time and temperature so as to enable that at least one fraction of the clay undergoes dehydroxilation and, thus, alters the clay's crystalline structure.

Also, according to this embodiment, thermal treatment may be carried out during periods of time that vary between 30 minutes and 12 hours, at temperatures varying between 600° C. and 1,200° C.; preferably temperatures between 700° C. and 900° C. and periods of time between 1 hour and 8 hours, the most suitable periods of time being between 1 hour and 5 hours.

It should be noted that in the preparation of any of the granules object of the present invention, prepared with any composition, the starting material is powdered raw materials. When needed, the raw materials are homogeneously dry mixed. This powder is then granulated, employing known granulation processes. The granulation process is important for the granules to acquire some of their cited properties and stability.

For the purposes of this invention, the expression “products of liquid to creamy (or pasty) consistency” relates to formulations that present viscosity varying from about 1 mPa·s to about 1,000 Pa·s. For comparative purposes, 1 mPa·s corresponds to aqueous solutions; shampoos and oils present viscosity in the order of 100 mPa·s to 1 Pa·s; hair conditioners present viscosity in the order of 1 Pa·s to 10 Pa·s; creams and dental creams present viscosity in the order of 100 Pa·s. It should be noted that these are referential values and may vary according to different possible formulations.

The granules of the present invention may further contain additives selected from polymers of animal, vegetable or marine origin, pigments, dyes or encapsulating agents, such as: fragrances, essential oils, vegetable oils, mineral oils, animal extracts, vegetable extracts or a mixture thereof.

As a function of the end products in which the granules of the present invention will be used, they present an average particle size varying from about 0.01 mm to about 6 mm.

According to the present invention, the granules are normally pigmented to be more visible and to contribute to the visual aspect of the formulations in which they are to be used, therefore, emphasizing its primary function of visual agents. The pigmentation process may occur at any time during the preparation process of the granules object of the present invention.

The granules according to the present invention are capable of maintaining their physical integrity in the formulations of personal care and/or home care products with liquid to creamy (or pasty) consistency, that is, that present viscosity varying from about 1 mPa·s to about 1.000 Pa·s, disintegrating only at the moment the end product is used, by employing friction, when applicable. Maintaining their physical integrity means not undergoing visual changes, specially the disaggregation of the granule, during its manipulation for use in formulations of personal care and/or home care products. Physical integrity is maintained due to a set of factors related to the use of thermal treatment in the modification of the physico-chemical properties of at least one fraction of the clay used as raw material in the production of the granule. The granules are also capable of maintaining their visual integrity, in spite of small increases in their size as a result of liquid absorption. This small swelling may occur in the cases wherein the granule is composed by a small percentage of dehydroxilated clay and a high percentage of non-dehydroxilated clay. It should be noted, however, that this swelling is normal, it may occur, but it is limited, that is to say, the granule undergoes a small swelling but it stops and does not compromise the granule stability, its visual aspect or its physico-chemical properties.

Below are data on the characterization of granules obtained according to the present invention. These data are a function of the raw materials employed in the granules preparation and their respective concentrations. Some parameters are within the following ranges and limits:
Moisture Content: maximum 25%;
pH (2% w/w aqueous suspension): varying from about 5 to about 10;
Apparent density: varying from about 0.01 g/cm³ to about 5 g/cm³;
Surface area: varying from about 0.5 m²/g to about 100 m²/g.

The following examples further illustrate the invention and, as such, are not to be considered as limiting the scope of protection of the invention.

### EXAMPLES

### Example 1

### Granules of the Present Invention

Table 1 illustrates some possible compositions of the granules of the present invention consisting of clay or clay and starch:

TABLE 1

| Granule | | | |
|---|---|---|---|
| reference | Non-thermally | Thermally treated | |
| number | treated clay(s) | clay(s) | Starch(es) |
| Granule 1 | 0% | 100% | 0% |
| Granule 2 | 0% | 33% | 67% |
| Granule 3 | 4.5% | 62.5% | 33% |
| Granule 4 | 25% | 50% | 25% |
| Granule 5 | 74% | 1% | 25% |
| Granule 6 | 0% | 1% | 99% |

In these examples, the clay thermal treatment consisted of heating it during 2 h at 800° C.

### Example 2

### High Integrity of the Invention Granules in Fluid Media

Stability tests with granules of different compositions, some of which are presented in Table 1 above, were conducted in laboratory.

Some formulations of personal care and home care products were prepared in the laboratory, in order to determine the concentration parameters of traditional ingredients in these formulations that would enable keeping the granules suspended and homogeneously dispersed in formulations from fluid to creamy (or pasty) consistency, for prolonged periods of time. Among the parameters tested in the formulations prepared, the following are below:

1. Surfactants: 10% to 30% by weight of anionic surfactant(s), based on the total weight of the formulation; 5% to 15% by weight of amphoteric surfactant based on the total weight of the formulation; 1% to 2% by weight of non-ionic surfactant based on the total weight of the formulation.

2. Thickeners: 3% to 8% by weight of acrylate copolymer based on the total weight of the formulation; or 0.3% to 0.5% by weight of carbomer based on the total weight of the formulation; or 1% to 2% by weight of xanthan gum to the total weight of the formulation.

3. Co-thickener: 0.1% to 0.5% by weight of NaCl in relation to the total weight of the formulation.

Besides the formulations above prepared in the laboratory, commercial products sold in the national and international retail markets have also been acquired for testing purposes. For all products it was possible to establish a correlation between the formulation of the medium employed in the test and the composition of the granules tested. Some of the commercial products tested are listed below:

National retail market: Seda Citric shampoo (Unilever—Brasil); Seda Selective shampoo (Unilever—Brasil); Seda Hidraloe conditioner (Unilever—Brasil); Palmolive antidandruff shampoo (Colgate Palmolive—Brasil); Woolite laundry detergent (Reckitt Benckiser—Brasil); Ypê Clear detergent (Química Amparo—Brasil); Confort Classic fabric softener with colagen (Unilever—Brasil); Sorriso dental cream with fluorine and calcium (Colgate Palmolive—Brasil); Colgate Tripla Ação mint flavor dental cream (Colgate Palmolive—Brasil), Close Up—Original Red dental gel (Unilever—Brasil).

International retail market: Suave liquid soap (Unilever—EUA); Equate Ocean Breeze liquid soap (Wal Mart—EUA); Tide Stain Brush laundry detergent with enzymes (Procter & Gamble—EUA), Gain Original Fresh laundry detergent with enzymes (Procter and Gamble—EUA), All Stainlifter laundry detergent (Unilever—EUA), Purex Original Fresh laundry detergent (Dial—EUA).

The viscosity of the products tested varied from about 2,500 to about 100,000 mPa·s.

Granule 1 presented stability in all media tested. Granules 2 and 3 showed stability in all media tested, except for the formulations containing enzymes, that is, laundry detergents Tide Stain Brush and Grain Original Fresh. The enzymes are developed for removing organic molecules from the medium in the laundry process, in the case of the cited products. The granules containing starch usually do not resist in this medium, since some of the target molecules of the enzymes used in the home care products are starches and other complex organic molecules. Granules 4, 5 and 6 showed to be specially suitable for personal care products.

All granules presented thermal stability and no granule degradation or disaggregation was observed in all media tested, at a temperature of about 45° C. during periods of up to 6 months (maximum monitored period). The granules with compositions presented in Table 1 remained stable even at temperatures higher than those used in the production process of personal care and home care products.

Sensitivity tests were carried out to compare the degree of exfoliation or abrasion of different formulations containing the granules. Based on the compositions of the granules presented in Table 1, it was determined that granule 1 presents strong abrasion and does not disintegrate with manual friction. Granules 2 and 3 have moderate abrasion, but dissolve with the use of friction, both being less abrasive than granule 1, and granule 3 being less abrasive than granule 2. Granules 4, 5 and 6 have a very soft abrasion, being less abrasive than granules 1, 2 and 3. Granules 4, 5 and 6 easily disaggregate with manual friction during use.

Having described examples of the invention with reference to its preferred embodiments, must be understood that the scope of the present invention embraces other possible variations, being limited solely by the appended claims, including the possible equivalents therein.

1. A granule composition for use in formulations of personal care and/or home care products with liquid to creamy or pasty consistency, characterized in that it consisting of a combination of clay and starch, wherein at least one portion of the clay is thermally treated with alteration of a crystalline structure of at least one fraction of the treated clay.

2. A granule according to claim 1, characterized in that the granule comprises from about 1% to about 99% by weight of clay based the total weight of the composition.

3. A granule according to claim 1, characterized in that said thermal treatment causes dehydroxilation of the clay and is carried out at a temperature range between 600° C. and 1,200° C. for a period of time from 30 minutes to 12 hours.

4. A granule according to claim 3, characterized in that the thermal treatment is carried out at a temperature range between 700° C. and 900° C.

5. A granule according to claim 3, characterized in that the thermal treatment is carried out for a period of time between 1 hour and 8 hours.

6. A granule according to claim 5, characterized in that the thermal treatment is carried out for a period of time between 1 hour and 5 hours.

7. A granule according to claim 6, characterized in that the clay is bentonite, vermiculite, kaolin, talc or mica.

8. A granule according to claim 7, characterized in that the clay is bentonite.

9. A granule according to claim 1, characterized in that the starch is selected from the group consisting of corn starch, cassava starch, and mixtures thereof.

10. A granule according to claim 9, characterized in that the starch is selected from the group consisting of natural starch, or modified starch, and mixtures thereof.

11. A granule according to claim 10, characterized in that the starch is pre-gelatinized.

12. A granule according to claim 1, characterized in that it further contains additives selected from the group consisting of polymers of animal, vegetable or marine origin, pigments, dyes and encapsulating agents.

13. A granule according to claim 1, characterized in that it has an average particle size varying in the dimension ranges contained from about 0.01 mm to about 6 mm.

14. A granule according to claim 1, characterized in that it has high integrity in media with viscosity between 1 mPa·s and 1,000 Pa·s.

15. A personal care and/or home care product having liquid to creamy or pasty consistency, characterized in that it comprises a granule as defined in claim 1.

16. A granule for use in formulations of personal care and/or home care products with liquid to creamy or pasty consistency, characterized in that it consists of clay, wherein at least one portion of the clay is thermally treated, with alteration of the crystalline structure of at least one fraction of the treated clay.

17. A granule according to claim 16, characterized in that said thermal treatment causes dehydroxilation of the clay and is carried out at a temperature range between 600° C. and 1,200° C. for a period of time from 30 minutes up to 12 hours.

18. A granule according to claim 17, characterized in that the thermal treatment is carried out at a temperature range between 700° C. and 900° C.

19. A granule according to claim 17, characterized in that the thermal treatment is carried out for a period of time between 1 hour and 8 hours.

20. A granule according to claim 19, characterized in that the thermal treatment is carried out at for a period of time between 1 hour and 5 hours.

21. A granule according to claim 16, characterized in that the clay is bentonite, vermiculite, kaolin, talc or mica.

22. A granule according to claim 21, characterized in that the clay is bentonite.

23. A granule according to claim 16, characterized in that it further contains additives selected from the group consisting of polymers of animal, vegetable or marine origin, pigments, dyes and encapsulating agents.

24. A granule according to claim 16, characterized in that the granule has an average particle size varying in the dimension ranges contained from about 0.01 mm to about 6 mm.

25. A granule according to claim 16 characterized in that it has high integrity in media with viscosity between 1 mPa·s and 1,000 Pa·s.

26. A personal care and/or home care product having liquid to creamy or pasty consistency, characterized in that it comprises a granule as defined in claim 16.

27. A granule according to claim 1, characterized in that it further contains additives selected from the group consisting of fragrances, essential oils, vegetable oils, mineral oils, animal extracts, vegetable extracts and mixtures thereof.
